(19) United States
(12) Patent Application Publication
(10) Pub. No.: US 2020/0029887 A1
(43) Pub. Date: Jan. 30, 2020

(54) METHOD OF ASSESSING WOUND CONTAMINATION AND INFECTION

(71) Applicant: ENVUE IMAGING INC., Houston, TX (US)

(72) Inventors: Edward P. BUCHANAN, Houston, TX (US); David KHECHOYAN, Englewood, CO (US)

(21) Appl. No.: 16/487,426

(22) PCT Filed: Feb. 20, 2018
(86) PCT No.: PCT/US2018/018763

§ 371 Date: Aug. 20, 2019

Related U.S. Application Data
(60) Provisional application No. 62/461,545, filed on Feb. 21, 2017.

(51) Int. Cl. A61B 5/00; A61K 49/00; G01N 33/569; G01N 21/64

(52) U.S. Cl. CPC A61B 5/445; A61B 5/0071; A61B 5/0077; A61B 5/742; G01N 2021/6439; G01N 33/56916; G01N 21/6456; G01N 21/6428; A61K 49/0058

(57) ABSTRACT

The present application discloses a method, using monoclonal antibodies or alternative fluorophore biomarkers, in combination with near-infrared fluorescence (NIRF) imaging, for assessment of the bacterial contamination and infection of an acute or chronic wound for prescribing medical treatments. The method includes applying a detection reagent containing at least one type of monoclonal antibody bonded to a fluorophore, wherein the monoclonal antibody binds to a unique receptor site of a bacteria in the wound and the monoclonal antibody is detected by a NIRF imaging system. The fluorescence signal of the wound is used to determine the concentration of bacteria in colony forming units (CFUs) to assess whether the wound needs further treatment or is ready for closure.

### PRIORITY

This application claims the benefit of U.S. provisional application No. 62/461,545 filed Feb. 21, 2017, the entire content of which is expressly incorporated herein by reference thereto.

### FIELD OF THE INVENTION

The present invention discloses a method for an assessment to detect and quantify bacterial contamination, colonization, and infection in human acute and/or chronic wounds that allows for the implementation of timely and specific treatments. The method preferably utilizes a system comprising pathogen-specific fluorophore-tagged monoclonal antibodies or alternative fluorophore biomarkers in combination with near-infrared fluorescence (NIRF) imaging system for this assessment.

### BACKGROUND OF THE INVENTION

Microbial contamination and/or infection is a common deterrent to normal human wound healing processes and leads to increased risk of morbidity and mortality in patients afflicted with wounds. Differential presence and relative invasiveness of bacteria in wounds may be characterized as contamination, colonization, and critical colonization. Contamination refers to the presence of bacteria in wounds in low concentration with low rates of replication. Colonization refers to presence of a higher concentration of bacteria in wounds with active replication and expansion of the bacterial population. Critical colonization refers to concentrations of bacteria in wounds near a threshold for an established, overt infection. (Landis, Chronic wound infection and antimicrobial use, Adv Skin Wound Care, November 2008, 21(11):531-40).

Higher wound tissue microbial bio-burden is clearly correlated clinically with delayed wound healing. Traditional guidelines for clinical wound healing indicate that tissue quantitative microbial count of greater than 10⁶ CFU (colony forming units) per gram of tissue is likely to lead to impaired wound healing. The threshold concentration of bacteria that leads to impaired wound healing may depend on the specific microorganism. (Landis, Chronic wound infection and antimicrobial use, Adv Skin Wound Care, November 2008, 21(11):531-40) Antimicrobial agents, such as antiseptics and antibiotics, are used in wound care to prevent or treat critical colonization and infection.

Human wounds that are afflicted by bacterial colonization often also harbor bacterial biofilm which consists of a population of tenaciously attached bacteria that are linked by an extra-cellular matrix. Presence of biofilm renders eradication of bacterial colonization or infection in wounds more difficult, leading to further impairment and delay in wound healing. (Percival et al., Biofilms and bacterial imbalances in chronic wounds: anti-Koch, International Wound Journal, Jun. 1, 2010, 7(3):169-175; Hall-Stoodley et al., Evolving concepts in biofilm infections, Cellular Microbiology, July 2009, 11(7):1034-1043)

Current, state-of-the-art diagnostic methods for detection and quantification of bacteria and fungi in wounds rely on traditional, laboratory-based techniques of gram stains and wound cultures. These methods are associated with a significant delay between the time when the wound is sampled and the time when clinically useful information is available to direct treatment. This delay in reporting accurate diagnostic information may be associated with: over-treatment with broad-spectrum antibiotics, promoting bacterial resistance; under-treatment because of choice of wrong antibiotic therapy; or a delay in reconstructive procedures.

Gram staining differentiates bacterial species based on the properties of the bacterial cell wall, which usually takes one to two hours to process in a laboratory. Gram staining provides limited information in identification of bacterial species and quantification of the microbial burden. Gram staining does not provide immediate results that may be utilized at the point of care to allow for timely changes in clinical management. Wound cultures allow for the identification of bacterial species, but require wound sampling, via a swab, fluid aspirate, or tissue biopsy, which typically take 3-7 days or longer to process before the final results are available. Quantitative wound cultures require an invasive tissue biopsy and specialized laboratory facilities with the capability to perform the testing. Both methods for bacterial culture require time and rely on specialized laboratory facilities to process the samples and are, therefore, inherently associated with a delay in providing the clinician with relevant diagnostic information.

Accordingly, there is a significant need for point-of-care technology to assess bacterial contamination and infection in wounds that would deliver timely testing results for making clinical decision at the time of the patient evaluation.

In this regard, Dacosta et al. (U.S. Pat, No. 9,042,967 B2) discloses a device and a method for fluorescence-based imaging and monitoring of a target composed of a light source emitting light for illuminating the target causing at least one biomarker associated with the target to fluoresce, and a light detector for detecting the fluorescence. The device and method is used for monitoring biological and non-biological substances, such as monitoring wound assessment. In particular, Dacosta et al. directly illuminate at least a portion of a wound and an area around the wound with a homogeneous field of light produced by a light source of a handheld device, wherein the light source emits at least one wavelength or wavelength band causing at least one biomarker in the illuminated portion of the wound and area around the wound to fluoresce; and then uses at least one optical filter to obtain optical signals emitted in response to illumination of the portion of the wound and the area around the wound for detection of the filtered optical signals in real time. Dacosta et al. thus uses intrinsic biomarkers for imaging. However, further critical improvements and advances in these procedures and methodology are necessary.

The present invention now addresses these needs and provides a viable improvement that has not been previously disclosed in the art. A point-of-care method for the assessment of wound contamination and infection is being developed to guide medical treatments of wounds at the time of examination.

### SUMMARY OF THE INVENTION

The present invention now provides a method for determining if a human wound is sufficiently devoid of bacterial contamination to be amenable to delayed primary closure or reconstruction. Equally importantly, it provides a method to assess the degree (concentration) and precise location of bacterial contamination that can guide further wound care or treatment, such as directed wound debridement, irrigation, or choice of dressing changes.

The present invention provides a method for determining if a subject's wound is ready for closure after treatment, which method comprises: washing the wound with a solution that comprises a fluorophore biomarker, such as a single monoclonal antibody or multiple monoclonal antibodies each bonded to a fluorophore, wherein the biomarker is configured for attachment to receptors on a single bacteria, bacterial species, groups of bacteria, or multiple bacteria; and exposing the wound after washing to near-infrared fluorescence (NIRF) imaging to observe fluorescence which indicates the presence of the bacteria or multiple bacterial species in the wound. The wound is ready for closure when the detected fluorescence of the bacteria indicates an amount that is not greater than a safe level, or a threshold, which the subject can tolerate without the closed wound becoming infected, but the wound would be further treated when the detected fluorescence of the bacteria indicates an amount that is unsafe for wound closure. In the latter situation, the wound is typically further debrided, irrigated, treated with topical medication, or other conventional wound care interventions.

The imaging is generally conducted with a NIRF imaging system after the treatment or further treatment to assess the presence of bacterial contamination to determine if the wound is ready for closure. In the method of the present invention, wherein the antibody is a monoclonal, multiple monoclonal or polyclonal antibody which attaches to the bacteria, and is provided in a detection reagent comprising a reporter system which includes the fluorophore is conjugated to the antibody, wherein the fluorescence of the wound is used to determine the concentration of bacteria in colony forming units (CFUs). The detection reagent may further comprise an irrigation solution comprising saline, polyvisol, lactated ringers, normal irrigants found during surgical procedures, or combinations thereof.

The fluorophore can attach through the antibody to a biomarker that identifies either a single bacterial species, bacterial group, or multiple bacterial types. A so-called “cocktail” of antibodies can be used, in which the detection system will be specific for multiple different types of bacteria, bacterial species or bacterial types; this would subsequently allow for the quantification and qualification of the bacterial landscape of a wound at the point of intervention.

The detection reagent is either premixed by the manufacturer, or is made by adding the reporter system to the irrigation solution immediately prior to treating the wound. The detection reagent may further comprise chemicals to enhance the affinity of the reporter system to bacterial species within a wound.

In one aspect, the method of the present invention may comprise a further washing, irrigation or debridement of the wound when the detected fluorescence indicates a level of bacteria that is greater than the safe level, and further exposing the wound to NIRF imaging after the completion of the further washing, irrigation or debridement to determine if the amount of bacteria is reduced to a safe level. In another embodiment, the safe level of bacteria is less than 10⁵ CFU per sq cm of a wound surface for wounds that contain no foreign objects or medical implants. The safe level or threshold may also be considered as 10¹ CFUs, when there is a medical need or necessity to retain foreign objects or medical implants in the wound. Also, the threshold should be an undetectable amount of bacteria or bacterial species when there is a medical need or necessity to ensure that the wound is without contamination for the optimal outcome of the patient.

Any one or more of a wide variety of bacteria can be detected by the present invention. In another aspect of the present invention, when the detected fluorescence indicates a level of bacteria that is greater than the safe level, the method of the present invention may further comprise: applying medication or procedures to the wound to reduce infection; applying medication or procedures to the wound to enhance wound healing, subjecting the wound to further sharp debridement, to further irrigation, to remove necrotic tissue, or to remove contaminated tissue; or applying stem cell therapy to enhance wound healing. The final result is to achieve a reconstructed wound, a closed wound, or a healed wound without further infection.

In one embodiment, the subject's wound to be evaluated is a chronic wound which fails to progress through normal stages of healing, while in another embodiment, the wound is an acute wound that results from surgery, trauma, diabetes, pressure, vascular insufficiency, burns, necrotizing soft tissue, or vasculitis.

The method of the present invention further comprises providing the near infrared fluorescence (NIRF) imaging from a hand-held device that includes a near-infrared (NIR) camera to detect low light fluorescence and a visible electronic display that provides information as to the presence of bacteria, the quantity of bacteria, and their topographical location. The level of fluorescence detected by NIRF imaging system will electronically be converted to be represented as a CFU count to assist the treating practitioner with important medical decisions regarding further treatment. The electronic display provides information in the form of color that indicates the level of bacterial contamination, showed on the display as fluorescence with a varying intensity depending on the level of fluorescence detected by the NIRF imaging system.

In one embodiment, the display provides a color which is representative of a particular type of bacteria present in the wound, provides different color combinations which are representative of multiple bacterial species, groups, or types present in the wound, or provides a color which is representative of a particular fluorophore associated with a specific antibody. Another aspect of the present invention is an improvement in a handheld device that includes a NIR camera, with the improvement comprising an electronic display that responds to fluorescence representative of an amount of bacteria present in a wound, wherein the display provides information as to the quantity and quality of microbial contamination, using the level of fluorescence detected by the NIR camera to correlate with bacterial quantity by intensity of fluorescence, and to correlate with bacteria type by color of fluorescence, to provide the practitioner with the information to determine whether or not a given wound is ready for closure or required further medical treatment to heal without the risk of delayed wound healing or infection.

In this embodiment, the wound being investigated can be of an acute or chronic type, and further medical treatment can be based on the discretion of the medical provider depending on the findings of the examination with the NIRF imaging system. Typical further medical treatments include irrigation, debridement, application of topical medications, application of topical antimicrobials, introduction of oral or intravenous antibiotics, application of temporary wound healing devices, application of temporary dressings, changing the types of dressings being applied, changing frequency of dressing changes, other wound healing modalities not afore-mentioned but commonly used in the medical profession for the treatment of both chronic and acute wounds, or combinations thereof.

In the handheld device of the present invention, the electronic display is used to effectively provide easily identifiable information about the bacterial status of the wound. The light emitting fluorescent color that is detected and displayed indicates whether the wound contains a safe level of bacteria or not. Multiple fluorescent colors are shown when the reporting system contains multiple fluorophore bonded monoclonal antibodies specific to different types of bacteria. The display thus provides a color representative of a particular type of bacteria that is present in the wound and the intensity of the displayed color quantitatively indicates the level of bacteria in the wound. Quantification can determine what the next steps of treatment for the wound should be, wherein the quantification can be extrapolated and represented as CFUs. Preferably, the handheld device of the present invention can be in the form of an electronic tablet or an electronic table attached to a larger module if the capabilities of the device cannot be contained within a single tablet. Further the device could contain a hand held probe attached to a screen that would display the topography of the wound and signals that are detected by the probe.

### BRIEF DESCRIPTION OF THE FIGURES

Further features of the inventive concept, its nature and various advantages will be more apparent from the following detailed description, taken in conjunction with the accompanying figures, wherein:

FIG. 1 shows a method to evaluate wound contamination of acute (surgical or traumatic) wounds through optical imaging.

FIG. 2 shows a method to evaluate wound contamination of established or chronic (clinic or hospital setting) wounds through optical imaging.

FIG. 3 shows a system for evaluating wound contamination of acute, established, or chronic wounds through optical imaging.

### DETAILED DESCRIPTION OF THE INVENTION

Throughout this description, the preferred embodiments and examples provided herein should be considered as exemplar, rather than as limitations of the present invention. The present invention discloses a method for an assessment to detect and quantify bacterial contamination, colonization, and infection in human acute and/or chronic wounds. It also provides a method to assess the degree (concentration) and precise location of bacterial contamination that can guide further wound care or treatment, such as directed wound debridement, irrigation, or choice of dressing changes. The method of the present invention preferably utilizes a system comprising pathogen-specific fluorophore-tagged monoclonal antibodies or alternative fluorophore containing components that attach to biomarkers in combination with near-infrared fluorescence (NIRF) imaging system for this assessment.

A point of care method for the assessment of wound contamination and infection is now developed to guide the medical treatment of wounds at the time of examination by utilizing the combination of near-infrared fluorescence (NIRF) imaging and pathogen specific monoclonal antibodies. The invention provides a method that allows medical practitioners to rapidly assess the bacterial environment of a wound to guide the clinical treatments effectively for more efficient wound healing. In addition, the method of the present application may be used for sampling of traditional microbiological quantitative culture analysis.

If bacteria are found in the wound, the wound can be further irrigated, debrided, or treated with topical antimicrobials and subsequently re-imaged to ensure elimination or substantial reduction of bacterial contamination. If the wound is largely free of bacteria and is otherwise amendable to closure, early surgical closure or definitive reconstruction may be achieved. The medical practitioner can make clinical decisions at the time of wound interrogation without exclusive reliance on protracted laboratory testing.

Thus, the methods of the present application can be used to aid clinical decision making in the operating room during formal surgical wound debridement or in determining the extent of debridement in the acute wound infection settings, such as surgical treatment for necrotizing soft tissue infection. The wounds could be interrogated at the bedside or in the clinical setting, to make key decisions in the timing for reconstruction of common wounds, such as those resulting from both chronic and acute conditions such as burns, trauma, diabetes, vasculitis and pressure. In the clinic setting, this method can be employed to determine the appropriate course of wound care. This can include further debridement, irrigation, more frequent dressing changes, less frequent dressing changes, topical antimicrobials, and all other wound care modalities that are pertinent to the treatment of wounds.

The process involves irrigating the open wound with a solution that includes one or an array of bacterial species-specific fluorescent reporter(s), preferably a monoclonal antibody that is configured to have affinity to a particular epitope on a specific bacterial species. The wound is then exposed to a light source part of the imaging system and the observed fluorescence signal is analyzed both qualitatively (presence/absence of signal and location of signal) and quantitatively (signal intensity). The qualitative analysis comprises determining whether and where bacterial presence is noted. The quantitative analysis comprises converting the fluorescent signal intensity to bacterial concentration, expressed either in absolute terms, such as colony forming units (CFUs) or as CFUs per unit surface area of the wound. The precise correlation between the fluorescent signal intensity and bacterial concentration may be different for different species of bacteria and may be related to the relative affinity of the monoclonal antibody to a specific bacterial epitope. This relationship between the fluorescent signal intensity and CFUs of bacteria per unit surface of the wound would be determined for individual bacterial species that are common human wound pathogens based on experimental data and previously established standards.

The wound is determined to be ready for closure or reconstruction when the detected intensity of the fluorescence signal, and, accordingly, the bacteria concentration, indicates a concentration that is not greater than an established threshold that is typically considered to be 10⁵ CFU per gram of tissue, above which a surgical site infection may develop or impaired wound healing may be noted. If the concentration is noted to be above the threshold concentration, additional treatment is implemented. The information regarding the precise location of the bacteria in the wound would guide further treatment of the wound in the form of debridement, irrigation, or specific wound care strategies or dressings to effect a further reduction in bacterial concentration and thereby lead to wound healing if all other conditions are optimized. If the detected concentration is below the safe threshold of 10⁵ CFU per gram of tissue, the wound may then undergo definitive surgical treatment (delayed primary closure or reconstruction).

The system would provide the practitioner with the ability to assess the degree of bacterial colonization of an acute or chronic wound at the point-of-care. The imaging system may be utilized in a diverse set of clinical settings. In the operating room, the system may be utilized to investigate an acute surgical wound or incision prior to closure, so as to prevent or reduce the risk of surgical site infections. It may also be utilized to guide debridement of an established wound prior to reconstruction or delayed primary closure. The wound may be investigated with the imaging system multiple times in the same operative setting until sufficient debridement of infected or colonized tissue is accomplished as evidenced by reduction of bacterial concentration to below a safe threshold level. The system may also be utilized in clinics, inpatient wards, or wound care centers. It would provide qualitative and quantitative information to the medical practitioner to guide further wound treatment or administration of systemic or topical antimicrobial agents. Further wound treatment may consist of but not be limited to bedside debridement, irrigation, whirlpool therapy, or application of specific dressings to effect removal of contaminated, colonized, or infected tissue. The preferred species of bacteria that may be detected by this imaging system include but are not limited to Staphylococcus aureus, Coagulase-Negative Staphylococci species, Enterococcus faecalis, Streptococcus pyogenes, Staphylococcus epidermidis, Pseudomonas aeruginosa, Serratia marcescens, Escherichia coli, Proteus mirabilis, Enterobacter species, Klebsiella pneumoniae, or combinations thereof that may represent polymicrobial contamination, colonization, or infection, but the system can detect other bacterial species that may cause human skin or soft tissue infection or are wound pathogens.

The method is applicable for both chronic and acute wounds. Chronic wounds are defined as those wounds which fail to progress through normal stages of wound healing. Acute wounds are defined as those either created as a result of a surgical operation, either elective or emergent, a surgical procedure, or traumatic accident resulting in a wound. The method (imaging system) may be utilized for acute and chronic wounds of other possible etiologies which may include but are not limited to trauma, diabetes, pressure, vascular insufficiency, burns, necrotizing soft tissue, or vasculitis, among others.

The method further comprises applying a wash reagent to the wound. The washing reagent may contain a quantity of commonly used irrigation media, including but not being limited to, normal saline, lactated ringers, polyvisol, or other solution, combined with a monoclonal antibody or an alternative fluorescent marker specific for a certain bacterial species. The monoclonal antibody is diluted in the irrigation solution prior to topical administration to either an acute or chronic wound. The irrigation solution containing the monoclonal antibody is applied copiously throughout the entire surface of the wound to ensure complete saturation of bacterial targets with the monoclonal antibody. The wound is then imaged with a hand-held or portable NIR (near-infrared) imaging device to qualitatively and quantitatively evaluate the location and concentration of bacteria based on the fluorescent signal intensity. The wound may then be further irrigated or debrided and re-imaged with the repeat application of the reagent solution and NIRF imaging. This cycle of imaging and wound treatment may be repeated until bacterial concentration is reduced to non-detectable levels or sufficiently reduced to below a pre-determined safe threshold concentration (for example, 10⁵ CFU). The washing reagent solution may contain either a single fluorophore labeling reagent, such as a monoclonal antibody, to target a single bacterial species or a combination of a fluorophore labeling reagents that may target a polymicrobial population of bacteria. Alternatively, the washing reagent solution may contain a cocktail of fluorophore labeling reagents specific to a certain group of bacteria such as gram-positive, gram-negative, or acid-fast bacilli, utilized in combination or individually. The decision as to which labeling reagent “cocktail” to utilize is left to the practitioner, based on previous microbiological data from the same wound and regional or hospital-specific epidemiologic prevalence of certain bacterial pathogens. The entire diverse bacterial population of a given wound may be imaged collectively with a polymicrobial “cocktail” or individual component bacteria may be detected in a step-wise fashion, whereupon one reagent is added to the wound at a time and the wound is subsequently imaged with a NIRF portable device.

For detection, the method further comprises NIR imaging from a handheld device, or with a hand held component that includes a camera to detect the fluorescence and a visible reporter, in the form of a fluorophore conjugated to a monoclonal antibody, that provides both quantitative and qualitative information in regards to the bacterial status of a given wound. The reporter emits a level of light intensity that is registered by NIR imaging system. The level of light intensity recorded by the imaging system provides the treating practitioner with quantitative and qualitative information in regards to the bacterial status of a given wound. Qualitatively, the absence or presence of bacteria in a given wound can be determined. If the reporting system contains multiple monoclonal antibodies for differing bacterial all with distinctly colored fluorophores, the types of bacteria can be determined. If the reporting system only contains one monoclonal antibody specific to a single bacteria or a group of bacteria, the amount of information will be limited to what is present in the reporting system. Quantitatively, the reporter system will have a level of fluorescence being emitted as determined by the level of bacterial contamination. The higher the level of contamination, the higher the level of fluorescence can be emitted by the reporter system and recorded by the imaging system. This level of fluorescence will be electronically converted into a CFU count for the treating practitioner, so that a clinical decision can be made at the point of care. The clinical decision will be directly related to the level of contamination detected. Further wound irrigation, wound debridement, application of topical antibiotics, etc., depending on the wound status, will all be considered appropriate to decrease the level of bacteria presence. The reporter system has the ability and potential to identify different bacteria with different fluorescent colors. Fluorophores come in many different colors. Differing colors can be used to either identify specific groups of bacteria or specific species of bacteria. Depending on the monoclonal antibodies which are conjugated to, individual fluorophores can be used to spectate a wound at the point of care. The most commonly used fluorophores in clinical use today include methylene blue (MB) and indocyanine green (ICG).

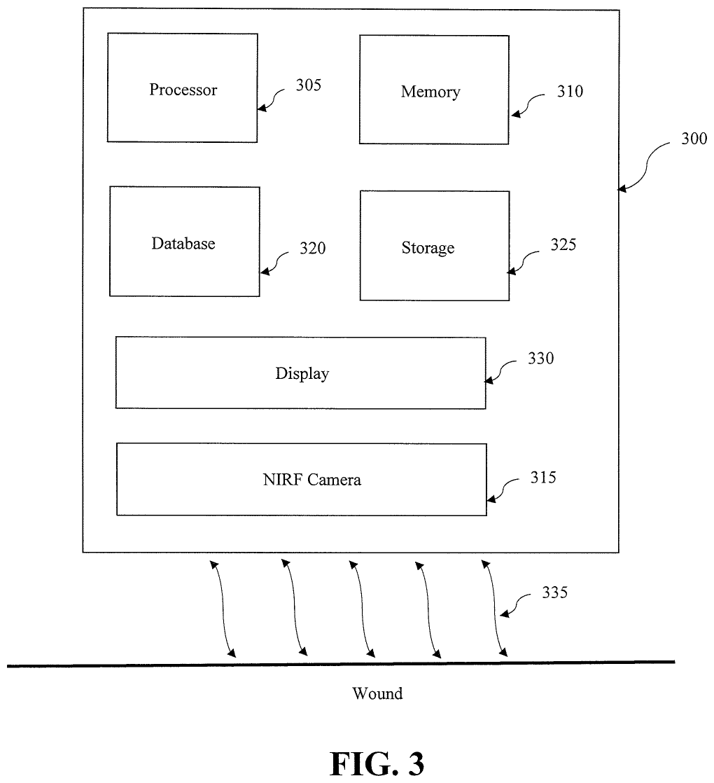

Another embodiment of the invention is directed toward the improvement of a handheld device 300 shown in FIG. 3 that includes a processor 305, a memory 310, a near infrared camera 315, a database 320, a storage 325, and a display 330. The camera 315 is configured to detect a level of fluorescence 335 which is representative of an amount of bacteria present in a wound, wherein the level of fluorescence 335 provides information as to whether or not the wound is ready for closure. The camera 315 is configured to detect fluorescence either with or without the assistance of a light source. In some embodiments additional light sources can be used to enhance the fluorescence detection. The camera 315 is electrically coupled to the processor 305 and transmits the detected fluorescence to the processor 305. The processor 305 can process the detected fluorescence based on the computer-executable instructions stored in the memory 310 and the data stored in the database 320. The data in the database 320 may include ranges of light intensity, ranges of wavelength, and codes representing different colors for each range. The data may further include bacteria information, antibody or fluorophore associated with each bacteria, wavelength of each bacteria and antibody or fluorophore, a minimum amount of intensity generated by a minimum amount of bacteria or antibody/fluorophore that can be detected by the processor for each bacteria and antibody or fluorophore, a conversion table for converting detected light intensity into a CFU count, and a translation table for translating the detected wavelength into the corresponding bacteria and antibody or fluorophore. In response to receiving the detected fluorescence, the processor loads the instructions in the memory to perform the processing which involves using the data stored in the database. The processor 305 determines the intensity of the detected fluorescence and the range of light intensity that includes the determined intensity. The processor 305 then determines the code or color that is associated with the determined range and instructs the display to output the associated color for the detected fluorescence. The processor 305 can also determine the wavelength of the detected fluorescence and the range of wavelength that includes the determined wavelength. The processor 305 then determines the code or color that is associated with the determined range and instructs the display 330 to output the associated color for the detected fluorescence.

The level of intensity of the detected fluorescence is controlled by the bacteria concentration in the wound. As it relates to detecting different types of bacteria within a particular wound, different types of fluorophores would have to be used for the different bacteria that were being investigated. For example, ICG coupled to a specific monoclonal antibody for bacteria A and methylene blue coupled to a specific monoclonal antibody bacteria B. This would allow for the detection system to register two different colors on the display and provide intensity readings to understand the level of each different bacteria present. The processor 305 can determine that bacteria A should displayed as green and bacteria B should be displayed as blue, due to their respective fluorophores. The exhibited wavelength detected is governed by the specific fluorophore or antibody used for that particular bacteria or by a specific gene or DNA in that particular bacteria. The processor 305 can also determine a first area in the wound that includes a high or unsafe concentration of bacteria A and that the first area should be displayed as red (instead of green), a second area in the wound that includes a low or safe concentration of bacteria A and that the second area should be displayed as green, a third area in the wound that includes a high or unsafe concentration of bacteria B and that the third area should be displayed as red or other color (instead of blue), and a fourth area in the wound that includes a low or safe concentration of bacteria B and that the fourth area should be displayed as blue. In some embodiments, the processor 305 can determine that an area should be dark green (higher intensity of green light or higher concentration of a bacteria) and another area should be light green (lower intensity of green light or lower concentration of the same bacteria). Since both areas are still green, they can still be considered as having a safe amount of bacteria for wound closure. In some embodiments, the darker color (dark green) can be considered as an unsafe concentration of the bacteria, instead of having to display that area as red. The processor 305 sends its color determinations to the display 330 and the display 330 shows the corresponding area with the respective determined color.

The camera 315, processor 305, memory 310, database 320, and display 330 operate in real-time to detect fluorescence and display the detected fluorescence as a color. Real time means that the system 300 performs an operation as it receives one or more inputs (e.g., a frame or an instruction from the camera). The system 300 operates such that the user of the system perceives the output (e.g., the color determined for an area) to be produced instantaneously (e.g., without appreciable delay perceived by the user) when the system receives an input. For example, the handheld device 300 can be directed to image a wound and display areas of the wound in different colors determined by the handheld device instantaneously. The system 300 can detect fluorescence and output the colors in real-time as the system moves over the wound.

The system or camera can record an image of the wound and areas in the wound that are displayed in colors determined by the system. For example, the image may show an area in the wound in green and another area in the wound in red. The system can also record a video of the wound and the procedures (or medical procedures) described above. The video can show change in color of the same area as the procedures are performed. For example, the video may show an area being irrigated changing from red to green gradually, or from a more intense signal to a less intense signal. This would indicate the level of bacteria present and being detected is decreasing as intervention measures are introduced to decrease the level of bacteria (e.g. irrigation, debridement, etc.) The image and video are saved in the storage for later retrieval. The image and video can be saved as evidence to show whether the wound before closure is clean or includes a level of contamination that is safe for closure. The processor can include a central processing unit (CPU), a graphics processing unit (GPU), a microprocessor (μP), an application specific integrated circuit (ASIC), a programmable logic array (PLA), a digital signal processor (DSP), a field programmable gate array (FPGA), a reduced instruction set computing (RISC) processor, or other types of processors. The processor controls the operations and performance of the system or handheld device. The processor can read and execute computer instructions that are stored on the memory. The memory can include random-access memory (RAM), cache memory, or other types of memory used for temporarily storing data. The database and storage can include read-only memory (ROM), erasable programmable read-only memory (EPROM), flash memory, solid state memory, ferroelectric RAM (FRAM), magnetoresisitve RAM (MRAM), hard disk drive, solid state drive. USB drive, floppy disk, magnetic tape, optical disc, or other types of memory and storage used for permanently storing data. The database and storage can be the same or different hardware devices. The database may also be a software database saved in the storage.

Suitable cameras for use in the present invention include Orca-ER, Model C9100-12, and C5985 cameras from Hamamatsu Photonic Systems, RTE/CCD-576 and TE/CCD-512SF cameras from Princeton instruments, Sony Cybershot DSC-T200 Digital Camera from Sony Corporation, and other standard analog or digital fluorescence cameras. As noted, the system includes instructions to convert the measured fluorescence intensity to quantitative values or colors. A preferred camera is disclosed U.S. Pat. No. 5,865,754 to Sevick. This particular camera has sufficient sensitivity to provide the florescence results needed for an accurate analysis. Other cameras may not be sufficiently sensitive if they are not designed to operate in a wound environment. As techniques exist for optimizing the camera to make it more sensitive, these newer designed, more sensitive cameras are deemed to fall within the scope of the present invention. In general, a camera that provides photon migration or photon enhancement, such as that of the Sevick patent, is preferred.

The display includes a screen and display circuitry for providing a display visible to the user. For example, the screen can be an LCD screen, LED screen, a touch screen, or a cathode ray tube (CRT). The display circuitry can include a coder/decoder (Codec) to convert digital data into analog signals and vice versa. For example, the display circuitry or other appropriate circuitry can include Codecs necessary to process the instructions or determined colors from the processor. The display circuitry also can include display driver circuitry, circuitry for driving display drivers, or both. The display circuitry can be operative to display content, e.g., application screens for applications implemented on the system or handheld device, the detected light intensity and wavelength, the bacteria and antibody or fluorophore associated with the detected light intensity and wavelength, and the CFU count associated with the e detected light intensity. The display circuitry can also receive and convert physical contact inputs from a touch screen, physical movements from a mouse or sensor, analog audio signals from a microphone, or other input. Alternatively, the display circuitry can be operative to provide instructions to a remote display or additional displays. Upon receiving instructions from the processor, the display can drive the pixels in the corresponding area in the wound or screen to display in the determined color.

The processor, memory, near infrared camera, database, storage, and display are electrically coupled by one or more communications buses. The bus can include a memory bus or memory controller, a peripheral bus, a serial bus, a PCI or PCIe bus, an accelerated graphics port, a process or local bus using any of a variety bus architectures. In some embodiments, some of these component may not be in the same system or handheld device. The camera may be used in the operating room, the computer containing the processor, memory, database, and storage may be in another room, and the display may be installed in yet another room. In this situation, the camera, computer, and display each may include a network interface and input/output (I/O) devices to communicate with each other. The network interface is configured to exchange data with an access point, a server, a computer, a camera, a display, or other devices via a communications network. The network interface is operative to interface with a communications network using any suitable communications protocol such as Wi-Fi, 802.11, Bluetooth, radio frequency systems such as 900 MHz, 1.4 GHz, and 5.6 GHz communication systems, infrared, GSM, GSM plus EDGE, CDMA, quadband, and other cellular protocols, VOIP, or any other suitable protocol. The network interface can include an Ethernet adapter (for wired connection), a wireless network adapter, a Bluetooth adapter, or other similar types of adapters. The communications network may also be established by using wires such as an optical fiber or Ethernet cable. The data and instructions can be transmitted and received between the processor, memory, near infrared camera, database, storage, and display via the network interface. Relevant data can be printed and stored in the patient's chart in the storage to demonstrate that the closure of the wound or surgical site was performed at an appropriate time. This can be further evidence of the quality of care that the patient is receiving which is important for insurance reimbursement and hospital performance evaluation.

The reporter system comes in the form of a monoclonal antibody specific to bacteria or groups of bacteria. The specific target for the monoclonal antibody will be bacteria specific and be highly based on binding affinity. It could include surface markers, metabolic products or by products, intracellular markers, or any other binding site commonly used for identification. The reporter system could be a monoclonal antibody specific for bacterial species or any marker, protein or molecule specific for bacterial species. A preferred handheld device is an electronic tablet. Such a device includes a NIRF camera and software that coverts fluoresence intensity to a quantitative value which can be displayed to inform the physician of the amount of bacteria remaining in different locations in the wound or surgical site in the patient. Additionally, the quantitative data can be converted to colors on the display with green indicating locations that have low amounts of bacteria, yellow indicating locations that have some but tolerable amounts of bacteria and red indicating locations where the bacteria content is too high such that further debridement or washing is needed before closure. The red locations can be further treated with the device then used to monitor improvement or cleaning progress to the point where all of the wound or surgical site exhibits the green or acceptable indication for closure. The screen display can be stored in the device as a record of the condition of the patient's wound or surgical site just prior to closure.

NIRF imaging requires a fluorophore that can be excited at near-infrared wavelengths of greater than 760 nm. NIR light is used to illuminate tissue surfaces and excite the fluorophore within the tissue. The resulting fluorescence is collected to form a 2-dimensional image that reflects the intensity and the location of the signal. NIRF imaging can detect fluorophore at variable depths below the tissue surface, depending on the brightness and sensitivity of the imaging device. A NIRF imaging system contains three core components, the light source to excite fluorophore, the optics to collect fluorescence and reject ambient and backscattered light, and the area detector to register the collected light.

Commonly used light sources in NIRF imaging include laser diodes, light emitting diodes (LED), and filtered lamp sources. Laser diodes provide the greatest sensitivity because they have the lowest backscattered excitation due to the use of interference filters. LEDs provide for a broader band of wavelengths, but require a number of integration together for milliwatts per square centimeter of incident light. Because filtered lamps have low light efficiencies, they are difficult to couple into an optical fiber and are not commonly used. Laser diodes and LEDs are the most used light sources in NIR imaging.

The most commonly used area detectors in NIRF imaging systems are charge-coupled device (CCD) detectors. They integrate the collection of photons over millisecond time frame. There are a variety of CCDs used in NIRF imaging today, including the front and back-illuminating CCDs (FCCD & BCCD), the electron multiplying CCD (EMCCD), and the intensified CCD (ICCD). The most commonly used CCD in the clinical setting today are the FCCD and the BCCDs. ICCDs and EMCCDs have an advantage over FCCD and BCCDs because they have the ability to enhance the signals that are being collected once they reach the area detector, which is beneficial in extremely low light environments. EMCCDs detectors enhance the incoming photon through collisional amplification. ICCD detectors utilize an image intensifier to amplify the incoming image before it is registered to the CCD. The intensifier consists of three parts: a photocathode, a microchannel plate, and a phosphor screen. Incoming light is converted into electrons by the photocathode which are then amplified by an electrical field near the microchannel plate and converted back into a light signal at the phosphor screen to be acquired by the CCD.

An ideal area detector in the clinical setting would have a low Quantum Efficiency (QE), with the ability to block any ambient light from being detected, and allowing only for detection of photons being emitted from the investigated source. Complementary metal oxide semiconductor (CMOS) is a newer area detector which is being used more frequently in the scientific setting. These scientific CMOS, or sCMOS, function differently than the CCD area detectors by utilizing an active pixel sensor system, in which every pixel combines a photodetector and its own active amplifier. This allows for more pixels on a single chip compared to the CCD cameras, with higher readout rates. EMCCD systems collect photoelectrons at each sensor and then transfer them to a readout where they are converted to current, multiplied and digitized by the internal electronics. CMOS sensors convert the photoelectron to voltage on the pixel which are then loaded into column-level amplifiers and analog to digital converters. This allows the CMOs camera to have an extremely fast operating time as well as no multiplication noise, commonly seen with the EMCCD sensor.

The system or handheld device can also be configured to display different lights that would indicate the results of the detection. For example, for a seriously high level of bacteria, a red light on the camera would indicate that closure of the wound should not be performed without further washing or treatment. For Mid-level results, i.e., bacteria that is likely tolerable, a yellow light would provide an indication that the wound is not as clean as it could be and that further attention may be necessary. Finally, for a very low detection of bacteria, a green light can indicate that the wound is acceptable for closure.

Instead of a light on the device, an electronic tablet screen or other display can be used to actually illustrate how much florescence is detected. As noted above, the screen can be calibrated to include a scale that illustrates the red/yellow/green colors, based on signal intensity, which can be highlighted as a summary of what is displayed as a further indication of the condition of the bacterial content of the wound. Further, the display may illuminate the different intensities of signal detected by the probe. The levels of intensity would correlate to level of bacterial contamination and would give the medical practitioner a better understanding of the levels of wound contamination at the time of evaluation.

The device utilizes conventional cameras that are capable of detecting NIRF and that can determine fluorescence intensity. As noted, these may include Orca-ER. Model C9100-12, and C5985 cameras from Hamamatsu Photonic Systems, RTE/CCD-576 and TE/CCD-512SF cameras from Princeton instruments, Sony Cybershot DSC-T200 Digital Camera from Sony Corporation, and other standard analog or digital cameras. As noted, these cameras would include software to convert the measured fluorescence intensity to quantitative values or colors. The preferred camera of Sevick U.S. Pat. No. 5,865,754 has been found to supply sufficient sensitivity to provide the florescence results needed for an accurate analysis. Furthermore, a provision for storage or transmission of the data is included. This is important for establishing and confirming the standard of care that the patient receives, as it demonstrates that the wounds were “clean” prior to closure.

An unexpected benefit of the present invention is that it is an important visual reminder to the physician of the condition of the wounds so that they can be extensively cleaned to prevent bacterial infection. The analysis of florescence data provides details that are not readily observable otherwise.

The present application discloses a method for assessment of the bacterial contamination and infection of a wound for prescribing medical treatments using monoclonal antibodies and a near-infrared fluorescence (NIRF) imaging system, wherein the monoclonal antibody binds to a biomarker of a bacteria in the wound and the monoclonal antibody is conjugated with a fluorophore or is detected with a fluorophore conjugated reporting system. The fluorescence of the wound is used to determine the concentration of bacteria in colony forming units (CFUs).

The present application also discloses a method for the evaluation and assessment of the bacterial contamination and/or infection of an acute or chronic wound for directing medical treatments which includes: applying a detection reagent, wherein the detection reagent comprises at least one monoclonal antibody, wherein the monoclonal antibody binds to a biomarker of a bacteria or group of bacteria in the wound, wherein the monoclonal antibody is conjugated with a fluorophore or is detected with a fluorophore conjugated reporting system; exposing the wound after applying the detection reagent to a near-infrared fluorescence (NIRF) imaging system to observe fluorescence, wherein the fluorescence of the wound is used to determine the presence of bacteria and the concentration of bacteria in colony forming units (CFUs).

In one aspect, the bacteria in the wound to be detected in the method for assessment of the bacterial contamination and infection of a wound in the present application is any bacteria known to cause contamination and or infection in human skin or the human body. These bacteria include but are not limited to Staphylococcus aureus, Coagulase-Negative Staphylococci species, Enterococcus faecalis, Enterococcus species, Streptococcus pyogenes, Staphylococcus epidermidis, Pseudomonas aeruginosa, Serratia marcescens, Escherichia coli, Proteus mirabilis, Enterobacter species, Klebsiella pneumonia, Streptococcus species, Staphylococcal species, Pseudomonas species, Serratia Species, Escherichia species, Proteus species, Enterobactor species, non-Enterobacteriaceae aerobes, Aeromonas species, Plesiomonas species, Erysopelothrix rhusiopathiae, Bacillus species, Klebsiella species, Haemophilus influenza, Haemophilus species, Enterobacter species, Fusobacterium nucleatum, Fusobacterium species, Prevotella melaninogenica, Prevotella species, Acinetobacter species, Citrobactor Species, Campylobacter species, Helicobacter species, Leptospiral species, Mycobacterial species, Salmonella species, Shigella species, Tsukamurella species, Nocardiaceae species, Cornybacterial species, Rhodococcal species, Vibrio species, Yersinia species, Peptostreptococcus species, Pasteurella species, Clostridial species, Bacteroides species, Xanthomonas species, Moraxella species, Acinetobacter species, Legionella species, Burkholderia species, Aeromonas species, Neisseria species, Serratia species, Eikenella species, Gram-positive anaerobes, Gram-negative aerobes, Gram-negative bacterial species, Gram-positive bacterial species, other anaerobic species, other aerobic species, or combinations thereof.

A diverse spectrum of contrast agents may be utilized to image bacteria in situ in an acute or chronic human wound. For the purposes of this invention, a contrast agent is a probe or a tracer that allows for direct or indirect imaging of bacteria in vivo. A direct imaging probe would directly image bacteria by binding specifically to components of the bacterial cell wall, cytoplasm, metabolic byproducts, and any other bacteria-specific epitope unique to bacteria. An indirect imaging probe would have specificity to an infectious process in the setting of a bacterial infection, including but not limited to, the host tissue response to infection, infection-specific biomarkers, or infection-specific pathways. Utilized in combination or independently, these direct or indirect probes (tracers) would allow for dynamic evaluation of the disease site (wound), enabling assessment of host-pathogen interactions and real-time, ongoing evaluation of response to treatment. The ideal set of molecular probes would be selected based on a collection of features that would optimize the imaging characteristics of the device associated with this method and the overall detection of the bacteria in wounds. These probe features may include, but are not limited to, low tissue absorbance; low tissue scattering; photostability; favorable signal-to-noise ratio; aqueous solubility; ease of conjugation (or chemical binding) to the targeting moieties, among others. The fluorophore probe may be administered to the wound conjointly with a fluorescence quencher which is chemically released off the primary probe by a target bacterial enzyme enabling the fluorescent signal to be detected. The fluorescence probes utilized in this method may fluoresce in the standard near-infrared spectrum (700-900 nm, e.g. NIR-I) and/or other region of the electromagnetic spectrum (900-1400 nm, e.g. NIR-II). Carbon nanotubes such as single-walled carbon nanotube (SWCN) may be utilized as a fluorescent probe for this application. The SWCN may be conjugated to a bacteriophage or a bacterial-specific antibody for specific targeting of a bacterial pathogen.

The about method also allows the clinician to address the following questions in a more timely and dynamic fashion:

(1) Based on the imaging of the wound, is the clinical presentation consistent with a bacterial wound infection, colonization, or only inflammation?

(2) What category of bacteria is responsible for the wound infection? Gram positive, Gram negative, or more specifically what species of bacteria?

(3) Is the current treatment (e.g., wound care, periodic debridement, topical or systemic antibiotic) effective or should the current treatment strategy be modified, continued, or de-escalated?

In the absence of the above information provided by the method of the present invention, the clinician may only rely on traditional microbiological or molecular biological tests performed in a standard or specialized laboratory, due to the inherent delay in reporting the result, which often lead to delay in diagnosis or misdiagnosis that may promote development of bacterial resistance and multi-drug resistant (MDR) pathogens. The probes would allow for quantification of the bacterial load, determination of Gram status or identification of the bacterial species, and ongoing monitoring of treatment progress.

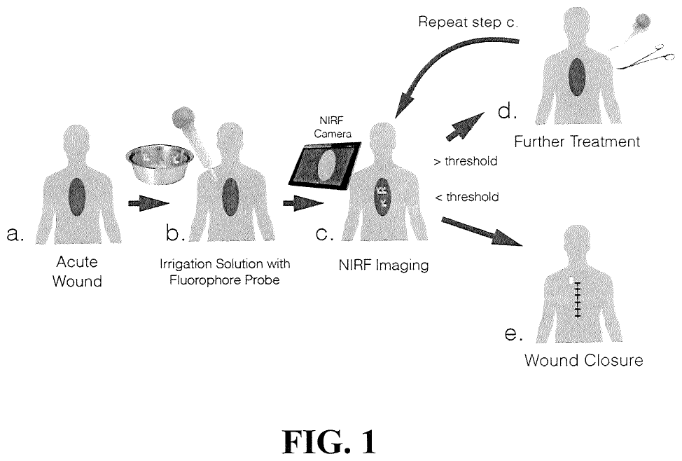

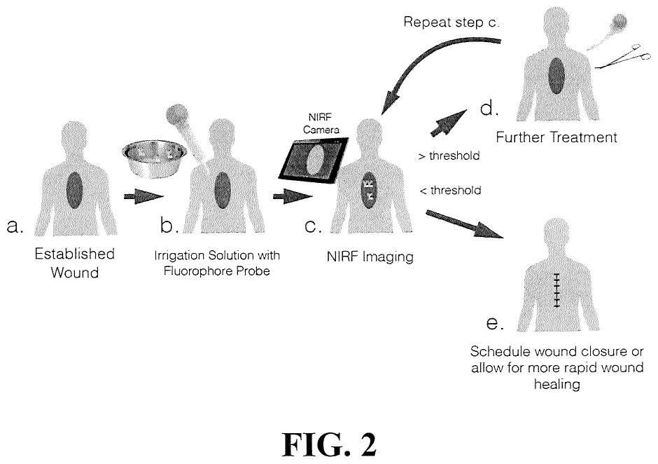

In the method of the present application, after the assessment of the bacterial contamination and infection of a wound, if the assessment is not below the threshold level, the prescribed medical treatment is to further irrigate, debride the wound, and/or apply topical antimicrobial specific to the microbe present. The wound is reimaging directly after the intervention is performed. The process is repeated until the minimum threshold level for microbial contamination is reached and definitive wound closure techniques or dressings are applied. If the wound is being treated in an acute setting, such as an operating room or emergency room, the appropriate closure techniques can be instituted. (FIG. 1) If this method is instituted in a wound care clinic or practitioners office, as is commonly seen in chronic wounds, the appropriate next steps to achieve a more fastidious wound closure can occur. These could include but are not limited to skin grafting techniques, application of dressings to allow for faster wound healing, application of ointments or creams, scheduling for operative intervention to achieve wound closure, instituting a new dressing, applying medication or procedures to enhance wound healing, debridement, reconstruction. (FIG. 2)

In these methods, the subject is an animal or preferably a human.

The wound may be a chronic wound, e.g., vasculitis, diabetic ulcer, pressure ulcer, which fails to progress through normal stages of healing, or it can result from an acute event, e.g. surgery, trauma, burns, necrotizing soft tissue. Please note that all causes of acute and chronic wounds have not been listed here, but the definition of an acute and chronic wounds as described in the introduction as the two major categories of human wounds and the types of wounds being treated by this method is emphasized.

In one aspect of the method of the present application for assessment of the bacterial contamination and infection of a wound for prescribing medical treatments and interventions, the detection reagent further comprises a medication to identify the presence of bacteria and bacterial species. The method comprises the application of a detection reagent, wherein the detection reagent comprises an irrigation solution, reporter system (one or multiple types of monoclonal antibodies) bonded to a fluorophore, and potentially other chemicals that allow for the swift and efficient binding of monoclonal antibodies to bacterial species present in the wound. The irrigation solution is normal saline, polyvisol, or lactated ringers, the normally occurring irrigation solutions found in operating rooms currently.

A preferred aspect of the method of the present application for assessment of the bacterial contamination and infection of a wound for prescribing medical treatments, the prescribed medical treatment is the closure of wound when the detected bacteria CFU indicates an amount that is not greater than a safe level, or threshold, which the body can tolerate and handle without the closed wound becoming infected.

In another aspect of the method of the present application for assessment of the bacterial contamination and infection of a wound for prescribing medical treatments, the prescribed medical treatment is applying medication or procedures to reduce infection or to enhance wound healing when the detected bacteria CFU indicates a level of bacteria that is greater than the safe level.

Another preferred aspect of the method of the present application for assessment of the bacterial contamination and infection of a wound for prescribing medical treatments and interventions, the method further comprises providing the NIRF imaging system from a hand-held device that includes a NIRF camera to detect low light fluorescence and an electronic display that provides the treating practitioner with information as to the bacterial status of the wound being examined, both quantitative and qualitative data. This display will immediately provide the practitioner the ability to understand whether the wound is at or near the threshold level for wound infection so that further decisions in regard to medical intervention can be introduced. The display typically provides information based on the signal that is emitted from the wound and detected by the NIRF imaging system. This signal is converted into an easily understood visible readout on the electronic display in the form of a light or color that indicates whether the wound contains the threshold level of bacteria. The display also provides the practitioner the ability to see the location of bacteria within the wound according to the wound topography. The detected signal from the wound is placed in context of the topographic location of the wound so that efforts to eliminate contamination and bacteria can be centered at the location in the wound where they are detected. The topographically display of the wound preferably be a real time, or as close to real time, camera display of the wound being examined. A topographical display of the wound with the location of bacteria based on the detected fluorescence signals from the NIRF imaging system will be provided to the practitioner in the electronic display on the hand-held device. The device will also convert the signal that is detected into CFU counts where applicable, so that the practitioner can understand where the bacteria are located in the wound and provide a directed and location specific treatment.

Further, the reporter system has the ability to identify single or multiple types of bacteria or bacterial species and allow for identification of individual species of bacteria in a given wound by color representation. The reporter system, which is added to the irrigation solution to create the detection reagent, can consist of a single monoclonal antibody specific for a single bacteria, bacterial species, or bacterial group, or multiple monoclonal antibodies types (cocktail), for the detection of multiple of species of bacteria, multiple individual bacteria, or multiple groups of bacteria. These monoclonal antibodies are of course bond to a fluorophore or have the ability to be detected by a fluorophore detecting system. This “cocktail” of monoclonal antibodies will be created and used depending on the indication needed. For example, if a treating practitioner wanted to speciate the wound which was treated immediately, they would use a reporter system that had the ability to identify multiple different types of bacteria within a given wound. The reporter system would differentiate the bacteria based on different color fluorescence that would be picked up by the NIRF imaging system and converted for viewing on the electronic viewer. Fluorophores come in many different colors, and these colors can be used to identify different bacterial species or groups within a given wound. Different color fluorophores would be bonded to different, species or bacterial specific, monoclonal antibodies. The monoclonal antibodies in a cocktail would all be specific to different receptors on the surfaces of differing bacteria, so that multiple different bacteria could be targeted during one investigation. Therefore, the proposed method has the ability to identify one single bacteria or multiple bacteria per use, and provides the practitioner with both quantitative and qualitative data in a topographical fashion about these bacteria.

Yet another aspect of the invention relates to the improvement of a hand-held device in a NIRF imaging system that provides this electronic display. The hand-held device may be in the form of an electronic tablet or a probe attached to a portable display. This electronic tablet or probe would be equipped with a sterile cover so that it could be used in the operating room setting. Further, the tablet could be attached to a larger module or console if all expected capabilities could not be housed in a single tablet. The purpose of the tablet would be so that the electronic display would provide for a true topographical representation of the wound in the form of electronic readout, and the signals that are being detected by the NIRF system are being displayed on the picture of the wound that is being displayed. This further provides the practitioner the ability to not only identify the presence of bacteria or bacterial species in a wound, but the location and a quantification, in the form of a CFU count, so that directed treatment can commence.

Experiments have been conducted to demonstrate that surface imaging of bacteria in vitro to determine the minimal detectable concentration of bacteria in CFUs (colony forming units). Near-infrared fluorescence (NIRF) imaging was utilized to detect the signal in real-time from a species-specific monoclonal antibody through a reporting system containing a fluorophore. The NIRF signal was correlated to the bacterial CFUs as determined by traditional laboratory microbiological analysis. This allowed the determination of imaging threshold in an in vitro model and the optimization of noise reduction, sensitivity and specificity of the imaging system. These initial in vitro experiments were done using Enterococcus faecalis as the detectable bacteria due to its virility and common occurrence in many types of human infections.

Enterococci has emerged as a significant human pathogen causing endocarditis and infections in bloodstream, wound, and urinary tract. Enterococcus faecalis is one of the most commonly occurring organisms in human skin wounds. The endocarditis and biofilm-associated pili (Ebp) of Enterococcus faecalis are constitutively expressed on the surface and are associated with the establishment of infections and the development of biofilms, playing the roles as virulence factors. Anchoring Ebp pili to the peptidoglycan surface of the bacterium allows pili to interact with the external environment. EbpA, EbpB, and EbpC are structural pilin components of pilus unit and EbpC is the major pilin protein (Pinkston et al., Targeting pili in Enterococcal pathogenesis, Infection and Immunity, 82(4):1540-1547, April, 2014). The IgG mAb-69, an anti-EbpC monoclonal antibody, has been validated as effectively localizing to the site of an established infection with E. faecalis in a Sprague-Dawley rat endocarditis model. This antibody is selected primarily because of its affinity to the recombinant EbpC antigen and its ability to recognize surface-displayed EbpC. (Pinkston et al., Antibody guided molecular imaging of infective endocarditis, Methods Mol. Biol., 2017, 1535:229-241).

Due to the similarities between human and porcine skin and the scale of wounds that may be introduced, the pig is a preferred animal model for wound healing and wound imaging studies. The experiments are conducted to demonstrate the surface imaging of bacteria in vivo in a porcine acute wound model with reproducible CFU threshold using a species-specific monoclonal antibody through a reporting system containing a fluorophore and a NIRF imaging system. In addition, the experiments are conducted to examine the imaging characteristics of the antibody-NIRF imaging system during different phases of wound healing and to assess wound healing outcomes as a function of bacterial contamination and infection. The NIRF signal intensity and location/distribution in the wound are recorded and correlated to the microbiological and histological analyses and results.

### EXAMPLES

The following examples illustrate the benefits and advantages of the present invention. In embodiments, a monoclonal antibody, mAb-69 was raised specifically against EbpC for the detection of E. faecalis to determine the minimal detection level of E. faecalis in an in vitro surface and an in vivo porcine acute wound model. mAb-69 was created at the University of Texas for the intended use of endocarditis identification. The anti-EbpC monoclonal antibody, mAb-69, has been demonstrated to bind to E. faecalis strain OG1RF specifically. (Pinkston et al., Antibody guided molecular imaging of infective endocarditis, Methods Mol. Biol., 2017, 1535:229-241)

### Example 1. Detection of Enterococcus Faecalis on an In Vitro Surface

The minimal detection level of E. faecalis was determined on an in vitro surface using monoclonal antibody labeling and NIRF FDPM (Near-infrared Fluorescence Frequency Domain Photon Migration) imaging system equipped with an intensified charge coupled device (ICCD) camera detector.

Various concentrations of E. faecalis OG1RF wild type cells and OG1RF ΔebpC deletion mutant cells (with the deletion of ebpC gene, a negative control) were labeled with mAb-69 (anti-EbpC monoclonal antibody) and detected with IRdye-conjugated secondary antibody or streptavidin using NIRF imaging, i.e. E. faecalis whole cell NIRF imaging. Reagents used in this example included mouse anti-EbpC mAb-69, biotinylated mAb-69, goat-anti-mouse Fc IgG-IR800, streptavidin-IR800, and PVDF-hydrophilic filter plate.

IRDye NIRF imaging was performed on the PVDF filter plate using the custom-built NIRF instrumentation. Various amounts of E. faecalis cells (incremental CFUs ranging from 10² to 10⁸ CFUs) were collected on the individual well membrane of the PVDF hydrophilic filter plate, followed by primary antibody binding (unlabeled or biotinylated anti-EbpC mAb-69) and secondary binding (goat-anti-mouse-IR800 or streptavidin-IR800). After extensive wash with PBST, membranes on the filter plate were let dry and imaged by an intensified CCD camera. The detection limit of the NIRF imaging using unlabeled mAb-69/goat-anti-mouse-IR800 is about 10⁶ E. faecalis cells. When biotinylated mAb-69/streptavidin-IR800 was applied, the minimum detectable cell number is about 10⁵ E. faecalis cells.

### Example 2. Detection of Enterococcus faecalis in an In Vivo Porcine Acute Wound Model

The experiments are performed to demonstrate the surface imaging of bacteria in vivo in a porcine acute wound model with reproducible CFU threshold using mAb-69 (anti-EbpC monoclonal antibody) and NIRF FDPM (Near-infrared Fluorescence Frequency Domain Photon Migration) imaging system equipped with a charge coupled device (CCD) camera detector.

Commercially-raised, pathogen-free, Yorkshire-cross pigs (20-25 kg, n=8) are utilized, which are the preferred animal model for wound studies because of physiological similarity to human wounds and the potential scale of wounds that may be introduced. Reagents used in the experiments include IR800 conjugated mouse anti-EbpC mAb-69, mouse anti-EbpC mAb-69 and goat-anti-mouse Fc IgG-IR800.

Full-thickness wounding and wound Inoculation:

On the day of operation (day 0), each animal undergoes induction of general anesthesia and intubation. Four, full-thickness, circular wounds in diameter of 3 cm are created with a trephine or scalpel per animal, followed by surgical excision of the wound plug. The four wounds are evenly distributed along the surface of the animals' backs, so as to minimize inter-wound cross-contamination and to maintain each surgical site as a distinct and independent wound environment.

On each animal, on day 0 of the study, the individual four wounds are inoculated with different concentrations of E. faecalis OG1RF in 10³ CFU/ml, 10⁵ CFU/ml, and 10⁷ CFU/ml respectively. Sterile saline is inoculated to one wound as control. The wounds are filled with a gauze pad saturated with 2 mL of E. faecalis OG1RF inoculum with respective CFU concentration or sterile saline. All wounds are sealed with an occlusive dressing (TEGADERM™3M), and the torso of each animal is wrapped with Kerlix gauze and Ace bandage. The initial dressings are maintained for 48 hours to allow for colonization or infection to be established.

Wound care and NIRF imaging:

The first dressing change is performed 48 hours following the operation (day 2). All subsequent daily dressing changes are performed with moist dressings consisting of Vaseline gauze. On day 2, each animal is briefly anesthetized for wound biopsy and the first NIRF imaging session. All wound dimensions are measured. Open wound areas are determined by measuring the length and width of the non-epithelialized wound portion with calipers and with wound tracing and imaging software, Aranz Silhouette Device (Aranz Medical).

Two small biopsies are acquired from each wound for quantitative microbiological cultures and for histological analysis. The tissue specimen for microbiology is placed in tubes containing 1 mL phosphate buffered saline, weighed, and homogenized. Dilutions are spot and/or spread plated on tryptic soy agar; and colonies are counted after overnight incubation at 37° C. The tissue specimen for history are fixed in formalin, processed, paraffin-embedded, and 5 μm sections are Gram stained or stained with hematoxylin and eosin.

The wounds are treated topically with a reagent containing IR800 conjugated mAb-69 or a reagent containing mAb-69 followed by a reagent containing goat-anti-mouse Fc IgG-IR800. Unbound antibodies are removed with the application of sterile saline. The NIRF signal intensity and location/distribution in the wound were recorded and correlated to the microbiological and histological analyses and results.

Based on the measured data, the wound would be ready for closure when the detected fluorescence of the bacteria indicates an amount that is not greater than a safe level or a threshold which the subject can tolerate without the closed wound becoming infected, while the wound would be further treated when the detected fluorescence of the bacteria indicates an amount that is unsafe for wound closure.

### Example 3. Detection of Enterococcus faecalis Across Different Phases of Wound Healing in an In Vivo Porcine Acute Wound Model

The same 8 Yorkshire-cross pigs that are utilized for Example 2 are followed continuously for Example 3. Example 3 is to examine the imaging characteristics of the mAb-NIRF imaging system during different phases of wound healing and to assess wound healing outcomes as a function of bacterial contamination or infection.

Following the biopsies and initial NIRF imaging performed on day 2, daily dressing changes are performed utilizing Vaseline gauze. On days 7, 14 and 21, each animal is briefly anesthetized for wound biopsy and a NIRF imaging session. All wound dimensions are measured. Open wound areas are determined by measuring the length and width of the non-epithelialized wound portion with calipers and with wound tracing and imaging software, Aranz Silhouette Device (Aranz Medical).

Two small biopsies are acquired from each wound for quantitative microbiological cultures and for histological analysis. The tissue specimen for microbiology is placed in tubes containing 1 mL phosphate buffered saline, weighed, and homogenized. Dilutions is spot and/or spread plated on tryptic soy agar; and colonies are counted after overnight incubation at 37 degree C. The tissue specimen for history is fixed in formalin, processed, paraffin-embedded, and 5 μm sections are Gram stained or stained with hematoxylin and eosin.

The wounds are treated topically with a reagent containing IR800 conjugated mAb-69 or a reagent containing mAb-69 followed by a reagent containing goat-anti-mouse Fc IgG-IR800. Unbound antibodies are removed with the application of sterile saline. The NIRF signal intensity and location/distribution in the wound were recorded and correlated to the microbiological and histological analyses and results. The detected fluorescence of the bacteria that indicates an amount that is not greater than a safe level or a threshold which the subject can tolerate without the closed wound becoming infected is recorded to demonstrate that the wound is suitable for closure and then the wound is closed. This can also be used to substantiate that the patient is receiving quality treatment in that the wound is not closed until bacteria levels are reduced to low and tolerable amounts.

As noted herein, a safe level is one that detects a level of bacteria that is less than 10⁵ CFU per sq cm of a wound surface for wounds that contain no foreign objects or medical implants. The safe level or threshold may also be considered as 10¹ CFUs when there is a medical need or necessity to retain foreign objects or medical implants in the wound.

It is to be understood that the present invention is not to be limited to the exact description and embodiments as illustrated and described herein. To those of ordinary skill in the art, one or more variations and modifications will be understood to be contemplated from the present disclosure. Accordingly, all expedient modifications readily attainable by one of ordinary skill in the art from the disclosure set forth herein, or by routine experimentation therefrom, are deemed to be within the true spirit and scope of the invention as defined by the appended claims.

What is claimed is:

1. A method for determining if a subject's wound is ready for closure after treatment, which comprises:
washing the wound with a solution that comprises a fluorophore associated with an antibody that is configured for attachment to receptors on a single bacteria, bacterial species, groups of bacteria, or multiple bacteria;
exposing the wound after washing to near-infrared fluorescence (NIRF) imaging to observe in real time fluorescence which indicates the presence of bacteria contamination in the wound from single or multiple bacterial species;
wherein the wound is ready for closure when the detected fluorescence of the bacteria indicates an amount that is not greater than a safe level, or a threshold, which the subject can tolerate without the closed wound becoming infected;
wherein the wound is further treated when the detected fluorescence of the bacteria indicates an amount that is unsafe for wound closure.

2. The method of claim 1, wherein the further treatment includes debriding, irrigating, treating with topical medication, or applying other wound care interventions and the method further comprises re-imaging the wound after the further treatment, with the imaging conducted with a NIRF imaging system after the treatment or further treatment to assess the presence of bacterial contamination to determine if the wound is ready for closure.

3. The method of claim 2, wherein the further treatments and re-imaging are repeated until the bacterial contamination in the wound is at or below a safe level for wound closure or normal wound healing.

4. The method of claim 1, wherein the antibody is a monoclonal, multiple monoclonal or polyclonal antibody which attaches to the bacteria, and is provided in a detection reagent comprising a reporter system which includes the fluorophore is conjugated to the antibody, wherein the fluorescence of the wound is used to determine the concentration of bacteria in colony forming units (CFUs).

5. The method of claim 4, wherein the detection reagent further comprises an irrigation solution comprising saline, polyvisol, lactated ringers, other normal irrigants used during surgical procedures, or combinations thereof.

6. The method of claim 4, wherein the fluorophore is attached to a component that binds to a biomarker that identifies a single bacterial species, bacterial group, or multiple bacterial types.

7. The method of claim 4, wherein the detection reagent further comprises chemicals to enhance the affinity of the reporter system to bacterial species within a wound.

8. The method of claim 5, wherein the detection reagent is created at the time of treating the wound, wherein the detection reagent is premixed by the manufacturer or is prepared by adding the reporter system to the irrigation solution immediately prior to treating the wound.

9. The method of claim 1 which further comprises a further washing, irrigation or debridement of the wound when the detected fluorescence indicates a level of bacteria that is greater than the safe level, and further exposing the wound to NIRF imaging after the completion of the further washing, irrigation or debridement to determine if the amount of bacteria is reduced to a safe level.

10. The method of claim 4, wherein the safe level of bacteria is less than 10⁵ CFU per square inch for wounds that contain no foreign objects or medical implants;
wherein the safe level or threshold to be considered is 10¹ CFUs, when there is a medical need or necessity to retain foreign objects or medical implants in the wound;
wherein the threshold is an undetectable amount of bacteria or bacterial species, when there is a medical need or necessity to ensure that the wound is without contamination for the optimal outcome of the patient.

11. The method of claim 1, wherein the bacteria to be detected is Staphylococcus aureus, Coagulase-Negative Staphylococci species, Enterococcus faecalis, Enterococcus species, Streptococcus pyogenes, Staphylococcus epidermidis, Pseudomonas aeruginosa, Serratia marcescens, Escherichia coli, Proteus mirabilis, Enterobacter species, Klebsiella pneumonia, Streptococcus species, Staphylococcal species, Pseudomonas species, Serratia Species, Escherichia species, Proteus species, Enterobactor species, non-Enterobacteriaceae aerobes, Aeromonas species, Plesiomonas species, Erysopelothrix rhusiopathiae, Bacillus species, Klebsiella species, Haemophilus influenza, Haemophilus species, Enterobacter species, Fusobacterium nucleatum, Fusobacterium species, Prevotella melaninogenica, Prevotella species, Acinetobacter species, Citrobactor species, Campylobacter species, Helicobacter species, Leptospiral species, Mycobacterial species, Salmonella species, Shigella species, Tsukamurella species, Nocardiaceae species, Cornybacterial species, Rhodococcal species, Vibrio species, Yersinia species, Peptostreptococcus species, Pasteurella species, Clostridial species, Bacteroides species, Xanthomonas species, Moraxella species, Acinetobacter species, Legionella species, Burkholderia species, Aeromonas species, Neisseria species, Serratia species, Eikenella species, Gram-positive anaerobes, Gram-negative aerobes, Gram-negative bacterial species, Gram-positive bacterial species, other anaerobic species, other aerobic species or combinations thereof.

12. The method of claim 1, wherein when the detected fluorescence indicates a level of bacteria that is greater than the safe level, the method further comprises: applying medication or procedures to the wound to reduce infection; applying medication or procedures to the wound to enhance wound healing;
subjecting the wound to further sharp debridement, to further irrigation, to remove necrotic tissue, or to remove contaminated tissue; or applying stem cell therapy to enhance wound healing in order to achieve a reconstructed wound, a closed wound, or a healed wound without further infection as a final result.

13. The method of claim 1, wherein the subject's wound to be evaluated is a chronic wound which fails to progress through normal stages of healing, or results from surgery, trauma, diabetes, pressure, vascular insufficiency, burns, necrotizing soft tissue, or vasculitis.

14. The method of claim 1 further comprising providing the near infrared fluorescence (NIRF) imaging from a hand-held device that includes a near-infrared (NIR) camera to detect low light fluorescence and a visible electronic display that provides information as to the presence of bacteria, the quantity of bacteria, and their topographical location, with the level of fluorescence detected by NIRF imaging system electronically converted to a CFU count to assist the treating practitioner in deciding whether the wound requires further treatment.

15. The method of claim 14, wherein the electronic display provides information in the form of color that indicates the level of bacterial contamination based on the intensity of fluorescence.

16. The method of claim 15, wherein the display provides a color which is representative of a particular type of bacteria present in the wound, provides different color combinations which are representative of multiple bacterial species, groups, or types present in the wound, or provides a color which is representative of a particular fluorophore associated with a specific antibody.

17. In a handheld device that includes a NIR camera, processor, memory storing computer instructions executable by the processor, database, display, the improvement being the device configured to respond to fluorescence representative of an amount of bacteria present in a wound, wherein the device provides information as to the quantity and quality of microbial contamination in a wound using the level of fluorescence detected by the NIR camera to correlate with bacterial quantity by intensity of fluorescence, the information presented (a) as a color indicator of how to proceed determined by the processor, memory, and the database; or (b) as a quantification of the approximate amount of bacteria that is present in the wound determined by the processor, memory, and the database, (c) or as a correlation of the bacteria type based on displayed color determined by the processor, memory, and the database, to provide the practitioner with the information to determine whether or not a given wound is ready for closure or required further medical treatment to heal without the risk of delayed wound healing or infection;
wherein the wound being investigated can be of an acute or chronic type;
wherein further medical treatment is based on the discretion of the medical provider depending on the findings of the examination with an NIRF imaging system, wherein the further medical treatment is irrigation, debridement, application of topical medications, application of topical antimicrobials, introduction of oral or intravenous antibiotics, application of temporary wound healing devices, application of temporary dressings, changing the types of dressings being applied, changing frequency of dressing changes, other wound healing modalities not afore-mentioned but commonly used in the medical profession for the treatment of both chronic and acute wounds, or combinations thereof.

18. The handheld device of claim 17, wherein the device provides a color on the display that indicates whether the wound contains a safe level of bacteria or not.

19. The handheld device of claim 17, wherein the intensity of the fluorescence quantitatively indicates the level of bacteria in the wound which can be extrapolated and represented as CFUs, and
wherein the device recommends or suggests whether the wound is ready for closure or not based on the level of bacteria detected in the wound by displaying processor determined colors on the display.

20. The handheld device of claim 17, in the form of an electronic tablet or an electronic tablet attached to a larger module if the capabilities of the device cannot be contained within a single tablet.
